(19) United States
(12) Patent Application Publication
(10) Pub. No.: US 2026/0256973 A1
(43) Pub. Date: Sep. 3, 2026

(54) VENTURI EFFECT ROOM FRAGRANCE DIFFUSER

(71) Applicant: Emil Dragos SAVULESCU, Milano (MI) (IT)

(72) Inventor: Emil Dragos SAVULESCU, Milano (MI) (IT)

(21) Appl. No.: 19/162,346

(22) PCT Filed: Mar. 15, 2023
(86) PCT No.: PCT/IT2023/000005

§ 371 Date: Sep. 4, 2025

(30) Foreign Application Priority Data
Mar. 7, 2023 (IT) 102023000004125

(51) Int. Cl. A61L 9/12

(52) U.S. Cl. CPC A61L 9/122; A61L 2209/11; A61L 2209/134

(57) ABSTRACT

An electrically powered room fragrance diffuser of the “Venturi effect” type is substantially cylindrical in shape with a convex upper base. The diffuser has a first cavity in a lower part thereof where a container enclosing the fragrance to be diffused is housed. The container is in communication with a second cavity interposed between the first cavity and the upper base of the diffuser. A pair of diametrically opposite fans sucks air into the second cavity through two respective inlet mouths at the side wall of the diffuser. The air sucked into the second cavity is blown by the fans into a conduit with an almost annular cross-section ending in an outlet mouth which is also annular. The outlet mouth is close to the upper base of the diffuser. The second cavity is shaped so that the air sucked by the fans loses pressure entering in the conduit so that, by the Venturi effect, the fragrance contained in the container is sucked into the second cavity forming a mixture with the air present therein to then be expelled into the environment surrounding the diffuser through the outlet.

### FIELD OF APPLICATION OF THE INVENTION

The present invention relates to the field of room fragrance diffusers. More precisely, the present invention relates to electrically-powered diffusers of the aforesaid type, i.e., diffusers in which the dispersion of a fragrance in the environment surrounding the diffuser occurs with a consumption of electricity.

Electrically-powered room fragrance diffusers are of different types depending on the mode with which the substance (generally in solid or liquid form) intended to emanate a fragrance is nebulized. In particular, in order to emanate a fragrance, the aforesaid substance can, by way of example, be heated, vibrated by means of an ultrasonic emitter or be exposed to a vacuum generated by Venturi effect by appropriately flowing an air stream close to the aforesaid substance.

Among the electrically-powered room fragrance diffusers, the present invention relates to diffusers utilizing the Venturi effect to nebulize the substance intended to emanate a fragrance.

### OVERVIEW OF THE PRIOR ART

Electrically-powered room fragrance diffusers utilizing the Venturi effect to nebulize the substance intended to emanate a fragrance generally comprise a fan by means of which air is sucked from the environment outside the diffuser and blown into a conduit ending in an outlet mouth. With reference to the direction in which the air blown by the fan travels through the conduit, the latter has a cross-section of decreasing width. By the Venturi effect, decreasing the cross-section of the conduit increases the air flow rate and decreases the pressure thereof. The substance intended to emanate a fragrance is appropriately placed in the diffuser so that the vacuum created by Venturi effect nebulizes or sucks the fragrance inside the conduit. The fragrance is thus mixed with the air blown by the fan and is expelled into the environment outside the diffuser through the aforementioned outlet mouth.

In the room fragrance diffusers of the aforesaid type, the outlet mouth of the conduit is usually circular and is generally placed at the top of the diffuser. Due to this, the mixture consisting of air and fragrance is introduced into the environment outside the diffuser, creating a sort of vertically directed scented “pinnacle.” However, the mixture of air and fragrance thus expelled from the diffuser disperses unevenly in the surrounding environment (said fragrance being blown exclusively upwards).

### Objects of the Invention

It is the object of the present invention to overcome the aforesaid drawbacks, indicating an electrically-powered room fragrance diffuser of the “Venturi effect” type which disperses the air and fragrance mixture more uniformly in the surrounding environment as compared to the known diffusers of the same type.

### SUMMARY AND ADVANTAGES OF THE INVENTION

The present invention relates to a room fragrance diffuser, said diffuser comprising:
a base at which said diffuser is placeable on a surface;
one or more peripheral walls;
a first cavity accessible, from the environment surrounding said diffuser, through a first opening placed at said base or said peripheral wall or at least one of said peripheral walls if more than one;
a second cavity at least partially travelable by an aeriform fluid,
   said second cavity being accessible, from the environment surrounding said diffuser, through at least one inlet mouth and at least one outlet mouth,
   said inlet mouth being placed at said peripheral wall or at least one of said peripheral walls if more than one,
   said outlet mouth being placed at said peripheral wall or at least one of said peripheral walls if more than one,
   said first cavity and said second cavity being separated from each other by at least one inner wall of said diffuser,
   said first cavity and said second cavity being in communication with each other through a second opening placed at said inner wall;
gas-operated means (i.e., means operating with an aeriform fluid) suitable for
   sucking air from the environment surrounding said diffuser into said second cavity through said inlet mouth
and for
   expelling in the environment surrounding said diffuser, through said outlet mouth, at least part of the air sucked into said second cavity,
so that at least part of the air sucked into said second cavity flows into the latter from said inlet mouth toward said outlet mouth,
said second cavity being shaped so that, when said gas-operated means suck air from the environment surrounding said diffuser into said second cavity, the air sucked into said second cavity, when traveling through the latter toward said outlet mouth, accelerates at at least one segment of said second cavity so that, at said segment, a decrease in air pressure occurs by Venturi effect,
said segment of said second cavity (at which said pressure decrease occurs) being sufficiently close to said second opening so that, when said gas-operated means suck air from the environment surrounding said diffuser into said second cavity, said decrease in air pressure causes a suction of air from said first cavity into said second cavity through said second opening;
control means connected to said gas-operated means and suitable for controlling both an operation and a stop thereof,
said diffuser being suitable for housing, at least partially, a container at said first cavity,
said container being at least partially housable in said first cavity and then removable therefrom through said first opening,
said container comprising a cavity delimited by at least one wall,
said cavity of said container being accessible through an opening placed at said wall of said container or at least one of said walls of said container if more than one,
said container being suitable for housing, in its cavity, an amount of a substance,
said substance housable in said cavity of said container being suitable for emanating a fragrance when:
   said substance is housed in said cavity of said container so as to comprise at least one free surface thereof (i.e., not matching with said one or more walls of said container) and
   air in contact with said free surface is sucked away from said free surface,
said fragrance, when emanated, being emanated by said substance at said free surface (Incidentally, if the substance suitable for emanating a fragrance is in the liquid state, the aforesaid free surface corresponds to the free surface of the substance),
said container being at least partially housable in said first cavity so that said opening of said container is in communication with said second opening of said diffuser so that:
   said cavity of said container is in communication with said second cavity of said diffuser through said opening of said container and said second opening of said diffuser and
   when said gas-operated means suck air from the environment surrounding said diffuser into said second cavity and said substance is housed in said cavity of said container, said decrease in pressure of the air sucked into said second cavity at said segment of said second cavity causes:
      a movement away from said free surface of air in contact therewith
      and therefore an emanation of said fragrance from said free surface
      and therefore a suction of said fragrance from said cavity of said container into said second cavity of said diffuser through said opening of said container and said second opening of said diffuser
      and therefore a mixing of said fragrance with at least part of the air sucked into said second cavity by said gas-operated means
      and therefore an expulsion into the environment surrounding said diffuser, through said outlet mouth, of said mixture including said fragrance and at least part of the air sucked into said second cavity,
where, according to the invention, said outlet mouth extends in length so as to form a closed path (so as to be outlined with a closed line) or, equivalently, said mouth comprises a series of openings placed at said peripheral wall, or at least one of said peripheral walls if more than one, and arranged side by side so as to form a closed path,
said outlet mouth being oriented so that, and said second cavity being shaped so that, when said gas-operated means suck air from the environment surrounding said diffuser into said second cavity, air sucked into said second cavity, or said mixture including said fragrance and at least part of the air sucked into said second cavity, is expelled into the environment surrounding said diffuser, through said outlet mouth, directed so as to give rise to a divergent flow away from said diffuser all around (i.e., in the entire surrounding space of) the latter.

The diffuser of the invention, by virtue of the particular shape of the outlet mouth and of the second cavity, disperses a mixture of air and fragrance into the surrounding environment in a more uniform manner as compared to the known diffusers of the same type. In fact, said mixture is not simply blown upwards, but in multiple, preferably horizontal directions diverging away from the diffuser.

Other innovative features of the present invention are disclosed in the following description and mentioned in the dependent claims.

According to an aspect of the invention, said outlet mouth is substantially annular,
said outlet mouth being oriented so that, and said second cavity being shaped so that, said flow with which air sucked into said second cavity, or said mixture Including said fragrance and at least part of the air sucked into said second cavity, is expelled into the environment surrounding said diffuser all around the latter when said gas-operated means suck air from the environment surrounding said diffuser into said second cavity, is substantially planar or at least partially frustoconical at least at a portion of said environment surrounding said diffuser adjacent to said outlet mouth.

According to another aspect of the invention, said outlet mouth is arranged horizontally when said diffuser is placed on a surface at said base,
said outlet mouth being oriented so that, and said second cavity being shaped so that, said flow with which air sucked into said second cavity, or said mixture including said fragrance and at least part of the air sucked into said second cavity, is expelled into the environment surrounding said diffuser all around the latter when said gas-operated means suck air from the environment surrounding said diffuser into said second cavity, is substantially, at least at a portion of said environment surrounding said diffuser adjacent to said outlet mouth:
   planar and substantially arranged horizontally or
   at least partially frustoconical with substantially vertical axis when said diffuser is placed on a surface at said base.

According to this aspect of the invention, the air and fragrance mixture is advantageously expelled from the diffuser into the surrounding environment thereof even more uniformly as compared to the known diffusers of the same type.

According to another aspect of the invention, said outlet mouth is delimited by a first edge and a second edge opposite to said first edge,
each of said first and second edges extending in length so as to form a closed path,
said second cavity, at least at a portion thereof adjacent to said outlet mouth, being interposed between a first wall and a second wall,
said first wall extending from said first edge to a third edge extending in length so as to form a closed path,
said second wall extending from said second edge to a fourth edge extending in length so as to form a closed path,
said first wall being at least partially surrounded by said second wall so that said portion of said second cavity corresponds to a gap (i.e., a “jacket”) interposed between said first wall and said second wall.

According to another aspect of the invention, said gap has a convergent trend away from said outlet mouth.

Incidentally, since the fragrance and air mixture sucked into the second cavity travels through the latter approaching the outlet mouth, the trend of the aforesaid gap approaching the outlet mouth is divergent, so as to facilitate giving a divergent trend to the flow of said mixture emitted by the diffuser not only away from the diffuser, but also, at least partially, from the surface on which the diffuser is placed at its base.

According to another aspect of the invention, said second cavity comprises a section at which said second cavity is in communication:
with the environment surrounding said diffuser through said inlet mouth,
with said first cavity through said second opening or
   when said container is housed at least partially in said first cavity, with said cavity of said container through said opening of said container and said second opening of said diffuser and
with said gap through a third opening (acting as the inlet mouth of said gap) delimited by said third and fourth edges,
said third opening, like said outlet mouth, extending in length to form a closed path,
said second opening and said third opening lying on opposite sides with respect to said section of said second cavity,
said section of said second cavity including, at least partially, said segment of
said second cavity in which, when said gas-operated means suck air from the environment surrounding said diffuser into said second cavity, said decrease in pressure of the air sucked into said second cavity occurs,
said third opening being included in said segment of said second cavity in which, when said gas-operated means suck air from the environment surrounding said diffuser into said second air cavity, said decrease in pressure of the air sucked into said second cavity occurs.

Incidentally, said segment of said second cavity in which, when said gas-operated means suck air from the environment surrounding said diffuser into said second air cavity, said decrease in pressure of the air sucked into said second cavity occurs and could partially coincide with (i.e., could be partially included in) said portion of said second cavity (i.e., said gap).

According to another aspect of the invention, said diffuser comprises at least two of said inlet mouths,
said section of said second cavity being in communication with the environment surrounding said diffuser through both said inlet mouths,
said two inlet mouths lying on opposite sides with respect to said section of said second cavity,
when said diffuser is placed on a surface at said base:
   said second opening lying at a lower height than that at which said third opening lies and
   each of said two inlet mouths lying at a lower height than that at which said third opening lies,
said gas-operated means comprising at least two vane impellers,
said two vane impellers lying on opposite sides with respect to said section of said second cavity,
each of said two vane impellers being opposite to one of said two inlet mouths.

According to another aspect of the invention, each of said two vane impellers is housed in said second cavity and is oriented so that the air sucked from the environment surrounding said diffuser, when sucked by said vane impeller into said second cavity, is at least partially sucked into said section of said second cavity and blown toward said third opening.

According to another aspect of the invention, said diffuser comprises reversible closing means of said outlet mouth (i.e., means suitable for occluding said outlet mouth and disoccluding the latter an indefinite number of times).

According to another aspect of the invention, said reversible closing means comprise:
a shutter including said first edge or said second edge;
movement means suitable for moving said shutter between at least:
   a first opening position, at which said first and second edges are not in contact with each other so that, when said gas-operated means suck air from the environment surrounding said diffuser into said second cavity, air sucked into said cavity, or said mixture including said fragrance and at least part of the air sucked into said second cavity, can exit from said diffuser through said outlet mouth and
   a second, closing position, at which said first and second edges are in contact with each other so as to occlude said outlet mouth so that, when said gas-operated means suck air from the environment surrounding said diffuser into said second cavity, air sucked into said cavity, or said mixture including said fragrance and at least part of the air sucked into said second cavity, cannot exit from said diffuser through said outlet mouth,
said control means being connected to said movement means and being suitable for controlling both an operation and a stop thereof,
said first wall extending from said first edge at least when said shutter is in
said first position (and not necessarily also when said shutter is in said second position),
said second wall extending from said second edge at least when said shutter is in said first position (and not necessarily also when said shutter is in said second position).

According to another aspect of the invention, said control means are suitable for preventing said gas-operated means from sucking air from the environment surrounding said diffuser into said second air cavity when said shutter is in said second position.

Advantageously, according to this aspect of the invention, when the diffuser is closed the gas-operated means do not suck air into the second cavity.

According to another aspect of the invention, said first wall at least partially delimits a third cavity surrounded by said gap with the interposition of said first wall,
said movement means being at least partially housed in said third cavity.

Advantageously, according to this aspect of the invention, the housing of the movement means in the third cavity optimizes the spaces inside the diffuser, reducing the overall footprint thereof.

According to another aspect of the invention, said diffuser comprises:
a top wall opposite to said base;
display means suitable for displaying visual information (i.e., capturable by a person through his/her sight) at said top wall so that said information is capturable by a person when observing said top wall,
   said control means being connected to said display means and being suitable for controlling them.

According to another aspect of the invention, said top wall is at least partially convex with the concavity facing said base.

Advantageously, according to this aspect of the invention, the information dis-played at said top wall is capturable in an optimal manner by an observer not only when he/she observes the aforesaid top wall with his/her head exactly thereabove (with reference to when the diffuser is placed on a surface at the base) but also when the observer is with his/her head slightly to the side with respect to the position taken by said observer with his/her head exactly above said top wall.

According to another aspect of the invention, said display means are suitable for displaying, at said top wall, information concerning the flow rate at which said gas-operated means suck air from the environment surrounding said diffuser into said second cavity through said inlet mouth.

According to another aspect of the invention, said container comprises at least one radio-frequency electromagnetic wave transceiver (e.g., an RFID tag) suitable for both receiving at least a first predefined signal and emitting, upon receiving said first signal, at least a second signal, also predefined,
said diffuser comprising radio-frequency electromagnetic wave transceiver means,
said control means being connected to said transceiver means and being suitable for controlling, by means of said transceiver means, an emission of radio-frequency electromagnetic waves,
said transceiver means being suitable for emitting first radio-frequency electro-magnetic waves corresponding to said first signal,
said transceiver means being suitable for receiving second radio-frequency electromagnetic waves corresponding to said second signal,
said container being housed in said first cavity so that, and said transceiver means being placed in said diffuser so that, when said transceiver means emit said first radio-frequency electromagnetic waves:
   said transceiver receives said first radio-frequency electromagnetic waves (corresponding to said first signal)
   and therefore said transceiver emits said second radio-frequency electro-magnetic waves (corresponding to said second signal) and
   said transceiver means receiving said second radio-frequency electromagnetic waves emitted by said transceiver,
said second radio-frequency electromagnetic waves emitted by said transceiver upon an emission of said first radio-frequency electromagnetic waves by said transceiver means encoding information concerning said fragrance being emanable by said substance housable in said cavity of said container,
said control means being suitable for decoding, from said second radio-frequency electromagnetic waves emitted by said transceiver, said information concerning said fragrance being emanable by said substance housable in said cavity of said container.

According to another aspect of the invention, said transceiver means comprise an antenna for transceiving radio-frequency electromagnetic waves arranged so that said antenna is at least partially wrapped around said container (and preferably, if present, around said neck of said container) when the latter is housed in said first cavity.

Advantageously, according to this aspect of the invention, by wrapping the antenna of the transceiver means around the container when the latter is housed in the first cavity, the probability is high that the transceiver means will be able to interact with the transceiver of the container regardless of the container portion at which the transceiver has been applied. In other words, according to this aspect of the invention, the risk that the transceiver means cannot interact with the transceiver of the container when the latter is housed in the first cavity of the diffuser is advantageously minimal.

According to another aspect of the invention, said display means are suitable for displaying, at said top wall, said information concerning said fragrance being emanable by said substance housable in said cavity of said container decoded by said second radio-frequency electromagnetic waves emitted by said transceiver upon an emission of said first radio-frequency electromagnetic waves by said transceiver means.

According to another aspect of the invention, said diffuser comprises electricity storage means,
said control means, said gas-operated means, said movement means, if present, said display means, if present, and said transceiver means, if present, being connected to said storage means for powering them,
said display means, if present, being suitable for displaying, at said top wall, information concerning the amount of electricity present in said storage means.

According to another aspect of the invention, said base is circular and said peripheral wall corresponds to, or said peripheral walls, if more than one, comprise, a cylindrical or frustoconical side wall connected to said base.

Advantageously, according to this aspect of the invention, the diffuser is housable, in a passenger car, at a seat commonly referred to as a “cup holder.”

According to another aspect of the invention, said diffuser comprises said container housed, at least partially, in said first cavity,
said container being housed in said first cavity so that said opening of said container is in communication with said second opening of said diffuser so that:
   said cavity of said container is in communication with said second cavity of said diffuser through said opening of said container and said second opening of said diffuser and
   when said gas-operated means suck air from the environment surrounding said diffuser into said second cavity and said substance is housed in said cavity of said container, said decrease in pressure of the air sucked into said second cavity at said segment of said second cavity causes:
      a movement away from said free surface of air in contact therewith
      and therefore an emanation of said fragrance from said free surface
      and therefore a suction of said fragrance from said cavity of said container into said second cavity of said diffuser through said opening of said container and said second opening of said diffuser
      and therefore a mixing of said fragrance with at least part of the air sucked into said second cavity by said gas-operated means
      and therefore an expulsion into the environment surrounding said diffuser, through said outlet mouth, of said mixture including said fragrance and at least part of the air sucked into said second cavity.

### BRIEF DESCRIPTION OF THE DRAWINGS

Further objects and advantages of the present invention will become apparent from the following detailed description of embodiments thereof and from the accompanying drawings, merely given by way of non-limiting explanation, in which:

FIG. 1 shows a front plan view of a room fragrance diffuser according to the present invention, with the fragrance emission mouth open;

FIG. 2 shows a diagrammatic straight section of the diffuser in FIG. 1 along plane A-A in FIG. 1;

FIG. 3 shows a front plan view of the diffuser in FIG. 1 with the fragrance emission mouth closed;

FIG. 4 shows a diagrammatic straight section of the diffuser in FIG. 3 along plane B-B in FIG. 3.

### DETAILED DESCRIPTION OF PREFERRED EMBODIMENTS OF THE INVENTION

Hereinafter in the present description, a figure may also be shown with reference to elements not expressly indicated in that figure but indicated in other figures instead. The scale and proportions of the various depicted elements do not necessarily correspond to the real ones.

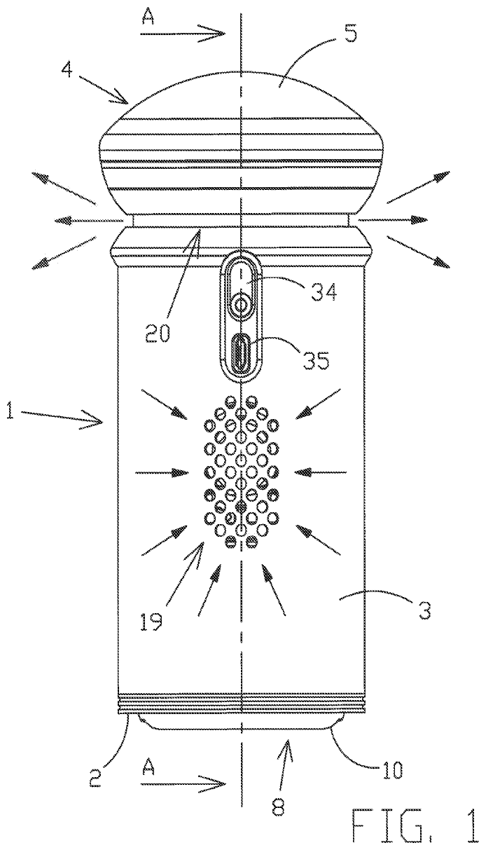

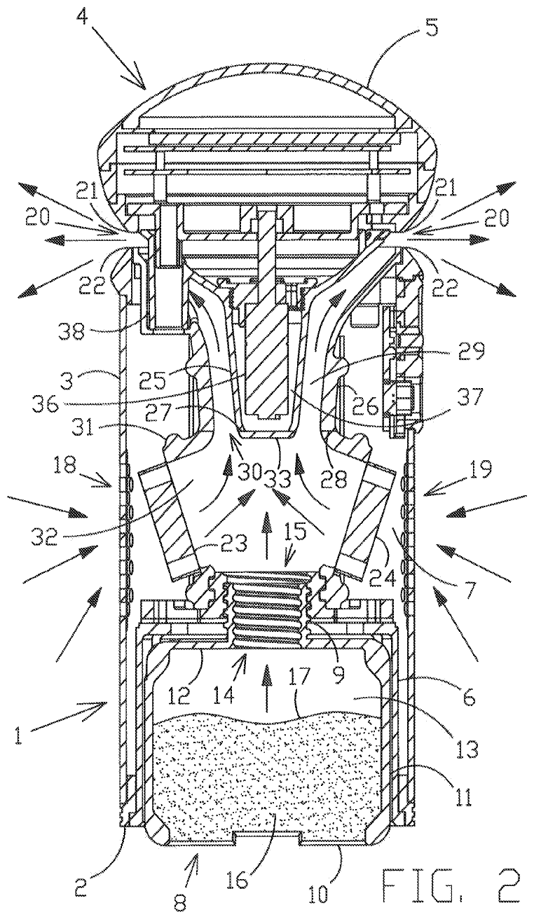

FIGS. 1 and 2 show a room fragrance diffuser of the invention, characterized by reference numeral 1. The diffuser 1 comprises a preferably circular base 2, at which the diffuser 1 is placeable on a surface. The diffuser 1 further comprises one or more peripheral walls connected to the base 2. More precisely, the diffuser 1 preferably comprises a preferably cylindrical or frustoconical side wall 3 rising from the edge of the base 2 preferably orthogonal thereto. The wall 3 is preferably surmounted by a sort of vault 4 preferably including a top wall 5 opposite to the base 2. The wall 5 is preferably at least partially convex with the concavity facing the base 2. The wall 5 is preferably shaped as a spherical cap the circular edge of which has the center preferably lying on the longitudinal axis of the wall 3 (i.e., the circular edge of which is preferably parallel to the edge of the base 2). Incidentally, the wall 3 being surmounted by the vault 4, the wall 3 is interposed between the vault 4 and the base 2.

The diffuser 1 comprises a first cavity preferably extending in said diffuser 1 from the base 2 toward the vault 4. Said first cavity is preferably cylindrical and coaxial to the wall 3, and has a height preferably almost equal to one third of the height of the wall 3. The aforesaid first cavity is accessible from the environment surrounding the diffuser 1 by means of a first opening preferably made at the base 2. Said first opening is preferably circular and concentric to the base 2, and preferably corresponds to the base of the cylinder as which the aforesaid first cavity is substantially shaped. The aforesaid first opening has an extension preferably close to that of the base 2.

The aforesaid first cavity is delimited, inside the diffuser 1, by a wall 6 separating said first cavity from a second cavity 7 of the diffuser 1. The first and second cavities of the diffuser 1 communicate with each other through a second opening 15 made in the wall 6. The aforesaid first and second openings are preferably mutually opposite. The diffuser 1, at the aforesaid first cavity, is suitable for housing a container 8. In FIGS. 1 and 2 the container 8 is shown housed at least partially in the aforesaid first cavity. The container 8 is preferably substantially shaped as a bottle including a substantially cylindrical body surmounted by a neck 9 also preferably cylindrical and coaxial to the aforesaid body. More precisely, the container 8 preferably comprises a preferably circular base 10 and a preferably cylindrical side wall 11 rising from the edge of the base 10 orthogonal thereto and ending in a top wall 12 also preferably circular and orthogonal to the wall 11 (and therefore preferably parallel to the base 10). The neck 9, substantially corresponding to a cylindrical side wall preferably orthogonal to the wall 12, has a cross-section of smaller diameter than that of the cross-section of the wall 11 (and therefore of smaller diameter than that of the wall 12). The base 10 together with the walls 11 and 12 delimit a cavity 13 accessible through an opening 14 made in the wall 12 at the base of the neck 9 lying on said wall 12. Therefore, the opening 14 is preferably circular and puts the cavity 13 in communication with the inside of the neck 9. Incidentally, the neck 9 is preferably coaxial to the wall 11.

When the container 8 is housed in the first cavity of the diffuser 1 (as shown in FIGS. 1 and 2), the wall 11 is preferably coaxial to the wall 3 (and therefore the base 10 and the wall 12 are preferably orthogonal to the wall 3) and the container 8 preferably almost completely occupies the aforesaid first cavity. Therefore, the wall 6 is preferably shaped like the walls 11 and 12 of the container 8 preferably together with the neck 9 thereof and is preferably opposite thereto (i.e., to the walls 11 and 12 and the neck 9) when the container 8 is housed in the first cavity of the diffuser 1. The wall 11 preferably but not necessarily has a height such as to protrude from the base 2 through the first opening of the diffuser 1 when the container 8 is housed in the first cavity thereof. As can be seen in FIG. 2, when the container 8 is housed in the first cavity of the diffuser 1 the neck 9 is housed in (i.e., crosses) the opening 15 made in the wall 6 so that the cavity 13 of the container 8 is in communication with the cavity 7 of the diffuser 1 through the opening 15 of the diffuser 1 (made in the wall 6), of the opening 14 of the container 8 (made in the wall 12) and the neck 9 thereof. Incidentally and with reference to when the container 8 is housed in the first cavity of the diffuser 8, the opening 15 of the diffuser 1 is delimited by the wall portion 6 opposite to the neck 9 of the container 8 and extends in the wall portion 6 opposite to the wall 12 of the container 8. Again incidentally and with reference to when the container 8 is housed in the first cavity of the diffuser 8, if the wall 11 has a height such as to protrude from the base 2 through the first opening of the diffuser 1, the latter is placeable on a surface at the base 10 of the container 8. This is equivalent to asserting that the diffuser 1 is placeable on a surface at the base 2 by means of the base 10 of the container 8. Still incidentally and with reference to when the container 8 is housed in the first cavity of the diffuser 8, the opening 14 of the container 8 is in communication with the opening 15 of the diffuser 1 by means of the neck 9.

The neck 9 is preferably externally threaded and the wall portion 6 opposite to the neck 9 of the container 8 when the latter is housed in the first cavity of the diffuser 1 is preferably internally threaded so as to couple to the external thread of the neck 9. In light of this, the container 8, at the neck 9, is preferably screwable to the wall 6 of the diffuser 1 at the opening 15. Incidentally, the container 8 is at least partially housable in the first cavity of the diffuser 1 and is then removable therefrom through the aforesaid first opening of the diffuser 1 (made in the base 2). The access opening to the first cavity (through which the container 8 is housable in the first cavity of the diffuser 1 and removable therefrom) could be made, instead of at the base 2, at the wall 3 or, more in general, at one of the peripheral walls of the diffuser 1. Incidentally, the container 8 being preferably screwable, at its neck 9, to the wall 6 of the diffuser 1 at the opening 15, the container 8 is reversibly connectable to (the wall 6 of) the diffuser 1, i.e., it is connectable thereto and disconnectable therefrom an indefinite number of times.

The container 8, at the cavity 13, is suitable for housing an amount of substance 16 so that said substance 16 comprises at least one free surface 17. The substance 16 (shown housed in the cavity 13 in FIGS. 1 and 2) is suitable for emanating a fragrance from the surface 17 when the air in contact with said surface 17 is sucked away therefrom. The substance 16 is, by way of example, a liquid and the surface 17 corresponds to the free surface of said liquid.

As will be disclosed in more detail below in the present description, the cavity 7 is at least partially travelable by an aeriform fluid. The cavity 7 is accessible, from the environment surrounding the diffuser 1, through at least one inlet mouth and at least one outlet mouth 20. There are preferably two inlet mouths and they are designated by reference numerals 18 and 19, respectively. The mouths 18 and 19 are preferably made at the wall 3, are preferably the same as each other and are preferably diametrically opposite. As can be seen in FIG. 1, each of the mouths 18 and 19 preferably comprises multiple openings of relatively small dimensions very close to one another, instead of consisting of a single opening. With reference to the position taken by the diffuser 1 when placed on a surface at the base 2 (possibly, as specified above, by means of the container 8), the opening placed at a lower height between the aforesaid multiple openings included in each of the mouths 18 and 19 preferably lies almost at the same height at which the portion of the wall 6 lies opposite to the wall 12 of the container 8 when the latter is housed in the first cavity of the diffuser 1. Each of the mouths 18 and 19 (i.e., the set of the aforesaid multiple openings included in each of the mouths 18 and 19) has a height (i.e., an extension in the direction of the longitudinal axis of the wall 3) preferably almost equal to one third of the height of the wall 3.

The mouth 20 is made in one of the peripheral walls of the diffuser 1 and is preferably interposed between the wall 3 and the vault 4. The mouth 20, unlike the mouths 18 and 19, preferably consists of a single opening extending in length so as to form a closed path (i.e., so as to be outlined with a closed line). More precisely, the mouth 20 is preferably substantially annular and is delimited by a first edge 21 and a second edge 22 opposite to the edge 21. Each of the edges 21 and 22 extends in length so as to form a closed path. In particular, the edges 21 and 22 are preferably the same as each other and each one preferably substantially corresponds to a circumference. The circumferences as to which the edges 21 and 22 are preferably substantially shaped are preferably parallel and are more preferably arranged orthogonally to a line passing through the centers thereof. In light of the foregoing, the mouth 20 is preferably shaped as the side surface of a straight cylinder having a height markedly less than the base diameter. When the diffuser 1 is placed on a surface at the base 2 (possibly, as specified above, by means of the container 8), the edges 21 and 22, and thus the mouth 20, are preferably arranged horizontally. In FIGS. 1 and 2 (in which the diffuser 1 is shown placed on a surface at the base 2 possibly, as specified above, by means of the container 8) the edge 21 is the upper edge of the mouth 20 and is preferably included in the vault 4, the edge 22 is the lower edge of the mouth 20 and is preferably included in the wall 3.

The diffuser 1 comprises means (previously qualified as “gas-operated”) by means of which, from the environment surrounding the diffuser 1, the air can be sucked into the cavity 7 through the mouths 18 and 19 and is expellable, at least in part, into the environment surrounding the diffuser 1 through the mouth 20 traveling through at least part of (i.e., flowing in) the cavity 7. Said means suitable for sucking air into the cavity 7 and at least in part expelling the latter into the environment surrounding the diffuser 1 preferably comprise a pair of vane impellers 23 and 24 (commonly defined “fans”) preferably housed in the cavity 7. More precisely, the impellers 23 and 24 are preferably the same as each other and are preferably opposite to the mouths 18 and 19, respectively. The impeller 23 is preferably in an intermediate position between the mouth 18 and the opening 15. The impeller 24 is preferably in an intermediate position between the mouth 19 and the opening 15.

As can be seen in FIG. 2, the cavity 7, at a portion thereof adjacent to (i.e., ending in) the mouth 20, is preferably interposed between a first wall 25 originating from the edge 21 (included in the vault 4), and a second wall 26 originating from the edge 22 (included in the wall 3). More precisely, the wall 25 extends from the edge 21 to a third edge 27 also extending in length so as to form a closed path, and the wall 26 extends from the edge 22 to a fourth edge 28 also extending in length so as to form a closed path. Each of the edges 27 and 28 preferably substantially corresponds to a circumference. The circumferences as to which the edges 27 and 28 are preferably substantially shaped are preferably parallel and are more preferably concentric (with the edge 27 of a smaller diameter than that of the edge 28). When the diffuser 1 is placed on a surface at the base 2 (possibly, as specified above, by means of the container 8), the edges 27 and 28 are preferably arranged horizontally. In light of the above, the wall 25 is at least partially surrounded by the wall 26 so that the aforesaid portion of cavity 7 corresponds to a gap 29 interposed between the walls 25 and 26. The edges 27 and 28 delimit an opening 30 (previously identified as “third opening” and extending in length to form a closed path) acting as an inlet mouth of the gap 29, preferably shaped as a circular crown and preferably arranged horizontally when the diffuser 1 is placed on a surface at the base 2 (possibly, as specified above, by means of the container 8). The gap 29 preferably has a diverging trend from the opening 30 toward the mouth 20 (i.e., it preferably has a converging trend away from the mouth 20).

To avoid any misunderstanding, the edge 21 preferably being included in the vault 4, asserting that the wall 25 extends from the edge 21 means that the wall 25 has an edge thereof, at the edge 21, opposite thereto. Similarly, the edge 22 preferably being included in the wall 3, asserting that the wall 26 extends from the edge 22 means that the wall 26 has an edge thereof, at the edge 22, opposite thereto.

The diffuser 1 preferably comprises an inner wall 31 which joins the wall 25 to the wall 6, wrapping the impellers 23 and 24, so as to delimit a further portion 32 of the cavity 7 (previously identified with the term “section” for greater clarity, so as to avoid confusion) in communication with the aforesaid first cavity of the diffuser 1 through the opening 15 and, when (as shown in FIG. 2) the container 8 is partially housed in the aforementioned first cavity of the diffuser 1, with the cavity 13 of the container 8 through the openings 14 and 15 and the neck 9. The portion 32 of the cavity 7 is further in communication with the gap 29 through the opening 30 and with the environment surrounding the diffuser 1 through the mouths 18 and 19 through the impellers 23 and 24. A preferably circular wall 33 closes the wall 25 thereon at the edge 27. Therefore, the latter also acts as an edge of the wall 33. The openings 15 and 30 are preferably opposite to each other. That is, they lie on opposite sides with respect to the portion 32 of the cavity 7. Even the impellers 23 and 24, as well as the mouths 18 and 19, lie on opposite sides with respect to the portion 32 of the cavity 7. When the diffuser 1 is placed on a surface at the base 2 (possibly, as specified above, by means of the container 8), the opening 30 preferably lies above the opening 15 and at a higher height than that at which both the mouths 18 and 19 and the impellers 23 and 24 lie.

Incidentally, the wall 31 could consist of an extension of the wall 26 beyond the edge 28 and the wall 33 could consist of an extension of the wall 25 beyond the edge 27, closing thereon.

Each of the impellers 23 and 24 is preferably oriented so that (i.e., the rotation axis of each of the impellers 23 and 24 is preferably inclined so that) the air sucked from the environment surrounding the diffuser 1 in the portion 32 of the cavity 7 is blown at least in part toward the opening 30. As can be seen in FIG. 2, the air sucked by the impellers 23 and 24 from the environment surrounding the diffuser 1, crosses the mouths 18 and 19 and the impellers 23 and 24 entering, at least in part, in the portion 32 of the cavity 7. From said portion 32 the air crosses the opening 30 and flows into the gap 29 until reaching the outlet mouth 20. From here the air exits the diffuser 1 returning to the environment surrounding the latter. In the passage from the portion 32 to the gap 29 the air travels through a segment of the cavity 7 in which, with reference to the air flow direction, a decrease of the cross-section of the “seat” of air flow occurs. The air sucked by the impellers 23 and 24 accelerates by traveling through the aforesaid segment of cavity 7. Said acceleration causes a decrease in the air pressure by the Venturi effect. The cavity 7, and in particular the portion 32 thereof together with the gap 29, is shaped so that (the aforesaid segment of cavity 7 where the aforementioned decrease in air pressure occurs is sufficiently close to the opening 15 so that), when the impellers 23 and 24 suck air from the environment surrounding the diffuser 1 into the cavity 7, and in particular into the portion 32 thereof, the decrease in air pressure occurring at the aforesaid segment of cavity 7 causes an air suction from the first cavity of the diffuser 1 into the portion 32 of the cavity 7 through the opening 15.

The portion 32 of the cavity 7 being, when (as shown in FIG. 2) the container 8 is partially housed in the aforementioned first cavity of the diffuser 1, in communication with the cavity 13 of the container 8 through the openings 14 and 15 and the neck 9, when: the container 8 is at least partially housed in the first cavity of the diffuser 1 as previously specified, the substance 16 is housed in the cavity 13 of the container 8 and the impellers 23 and 24 suck air from the environment surrounding the diffuser 1 into the cavity 7, and in particular into the portion 32 thereof, the decrease in air pressure occurring at the aforementioned segment of cavity 7 causes: a movement away from the surface 17 of air in contact therewith, therefore an emanation of a fragrance from the surface 17, therefore a suction of said fragrance from the cavity 18 of the container 8 in the portion 32 of the cavity 7 of the diffuser 1 through the openings 14 and 15 and the neck 9, therefore a mixing of the aforesaid fragrance with at least part of the air sucked into the portion 32 of the cavity 7 by the impellers 23 and 24, and therefore an expulsion into the environment surrounding the diffuser 1, through the mouth 20, of the aforesaid mixture including the fragrance and at least part of the air sucked into the portion 32 of the cavity 7.

Incidentally, the opening 30 is included in the aforementioned segment of the cavity 7 at which the air, when sucked into the portion 32 of the cavity 7 by the impellers 23 and 24, undergoes a decrease in pressure flowing toward the mouth 20. Therefore and again incidentally, the aforesaid segment of the cavity 7 at which the air, when sucked into the portion 32 of the cavity 7 by the impellers 23 and 24, undergoes a pressure decrease flowing toward the mouth 20 is at least partially included in the portion 32 of the cavity 7. Still incidentally, the aforesaid segment of the cavity 7 at which the air, when sucked into the portion 32 of the cavity 7 by the impellers 23 and 24, undergoes a pressure decrease flowing toward the mouth 20 could partially coincide with (i.e., could be partially included in) the gap 29.

The mouth 20 is oriented so that, and the cavity 7, at least at where the air flows when sucked in by the impellers 23 and 24, is shaped so that, when the impellers 23 and 24 suck the air from the environment surrounding the diffuser 1 in the portion 32 of the cavity 7, the sucked air, or the mixture including the fragrance emanated by the substance 16 and at least part of the air sucked by the impellers 23 and 24, is expelled into the environment surrounding the diffuser 1, through the mouth 20, directed so as to give rise to a divergent flow away from the diffuser 1 all around the latter, so that, when the container 8 is at least partially housed in the first cavity as specified above and the substance 16 is housed in the cavity 13 of the diffuser 1, the aforesaid mixture disperses in the environment surrounding the diffuser 1 more uniformly as compared to the known room diffusers of the same type. The aforesaid divergent flow away from the diffuser 1 all around the latter is preferably substantially planar or at least partially frustoconical at least at a portion of the environment surrounding the diffuser 1 adjacent to the mouth 20. More precisely, when the diffuser 1 is placed on a surface at the base 2 (possibly, as specified above, by means of the container 8), the aforesaid divergent flow away from the diffuser 1, at least at the aforesaid portion of the environment surrounding the diffuser 1 adjacent to the mouth 20, is preferably substantially planar and substantially horizontally arranged, or is preferably substantially at least frustoconical with a substantially vertical axis.

Incidentally, asserting that the aforesaid divergent flow away from the diffuser 1 is preferably substantially planar or at least partially frustoconical at least at a portion of the environment surrounding the diffuser 1 adjacent to the mouth 20 means that the aforesaid flow is preferably substantially planar or at least partially frustoconical at least immediately at the exit from the mouth 20.

Incidentally, the mouth 20 is for example equivalent to (i.e., instead of extending in length so as to form a closed path, it could also comprise) a plurality of openings made in one or more peripheral walls of the diffuser 1 and arranged in sequence at a relatively short mutual distance (i.e., a series of openings arranged side by side) so as to give rise to the closed path to which the mouth 20 gives rise extending in length. What it detects is that the mixture including the fragrance emanated by the substance 16 and at least part of the air sucked by the impellers 23 and 24 is emitted into the environment surrounding the diffuser 1 all around (preferably circumferentially to) the latter, generating a divergent flow away from the diffuser 1.

The diffuser 1 comprises a control unit connected to the motors which rotate the impellers 23 and 24 and suitable for both actuating and stopping the latter. Preferably, the control unit is also suitable for adjusting the rotation speed of the impellers 23 and 24 about its axis. A user of the diffuser 1 can operate or stop the impellers 23 and 24 (i.e., the motors which rotate them), by means of the aforesaid control unit, preferably by means of one or more buttons 34 preferably placed at the wall 3 (obviously so as to be pressable from outside the diffuser 1) preferably between one of the mouths 18 and 19 and the mouth 20. The control unit and the motors of the impellers 23 and 24 are preferably electrically powered by means of an electric accumulator housed inside the diffuser 1. The electric accumulator is preferably rechargeable by connecting it to the electrical network or to a source of electric current by means of a cable. To this end, the diffuser 1 is preferably provided with a socket 35, preferably of the known “USB” type, preferably placed close to the button 34 (or buttons 34, if more than one).

As previously mentioned, the base 2 of the diffuser 1, as well as the base 10 of the container 8, is preferably circular and the side wall 3 is preferably cylindrical or frustoconical. By virtue of this, appropriately sizing the diffuser 1, the latter, in addition to being for example usable in a house, is housable, by way of example, in a passenger car at a seat commonly referred to as a “cup holder.”

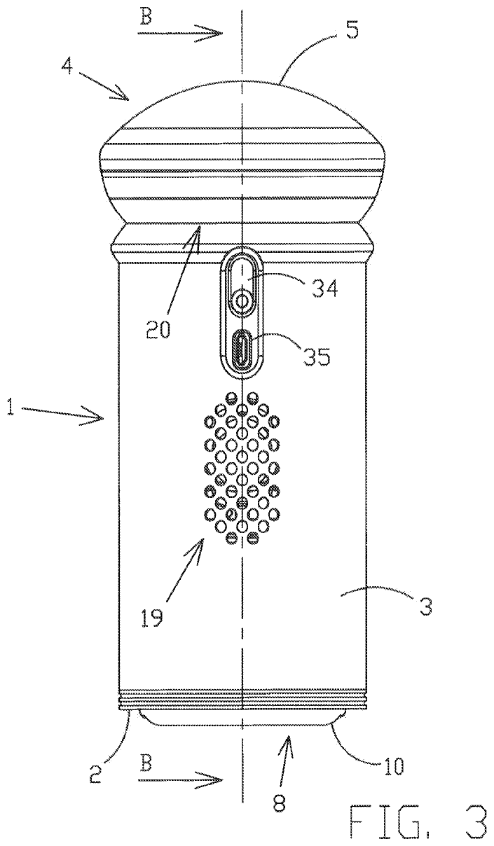

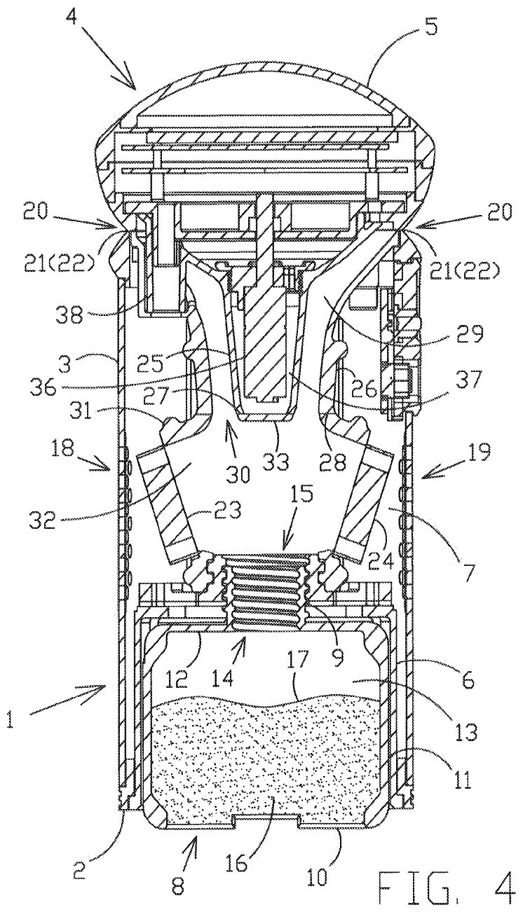

The mouth 20 of the diffuser 1 is preferably closable when the diffuser 1 is not in use. To this end, the diffuser 1 preferably comprises means suitable for reversibly closing the mouth 20. More precisely, the diffuser 1 preferably includes an actuator 36 suitable for moving with respect to the wall 3, and preferably for translating the vault 4 in the direction of the longitudinal axis of the wall 3, acting as a shutter, between at least:
a first opening position (shown in FIGS. 1 and 2), at which the edges 21 and 22 are not in contact with each other so that, as described so far, when the impellers 23 and 24 suck air from the environment surrounding the diffuser 1 in the portion 32 of the cavity 7, the sucked air, or the mixture including the fragrance emanated by the substance 16 and at least part of the air sucked by the impellers 23 and 24, can exit from the diffuser 1 through the mouth 20 and
a second closing position (shown in FIGS. 3 and 4), at which the edges 21 and 22 are mutually in contact, preferably matching each other, so as to occlude the mouth 20 so that when the impellers 23 and 24 suck air from the environment surrounding the diffuser 1 in the portion 32 of the cavity 7, the sucked air, or the mixture including the fragrance emanated by the substance 16 and at least part of the air sucked by the impellers 23 and 24, cannot exit from the diffuser 1 through the mouth 20.

The control unit prevents the impellers 23 and 24 from sucking air from the environment surrounding the diffuser 1 when the vault 4 is in the closed position.

As can be seen in FIGS. 2 and 4, the wall 25, together with the wall 33, delimits a third cavity 37 surrounded by the gap 29 with the interposition of the wall 25 and with the concavity facing the vault 4. The preferably linear actuator 36 is preferably housed in the cavity 37. The walls 25 and 26 are preferably integral with the wall 3. A translation of the vault 4 from the opening position to the closing position thus results in a translation of the edge 21 toward the edge 22 preferably without this resulting in any translation of the wall 25 toward the wall 26. In light of the foregoing, the wall 25 extends from the edge 21 only when the vault 4 is in the opening position. Upon a translation of the vault 4 from the opening position to the closing position, the wall 25 partially penetrates the vault 4 at least at the edge of the wall 25 which is opposite to the edge 21 when the vault 4 is in the opening position.

The diffuser 1 could also comprise one or more guides 38 which constrain the vault 4 to translate, with respect to the wall 3, as imposed by the actuator 36. The guides 38 are preferably interposed between the vault 4 and the wall 26 (integral with the wall 3). Although the guides 38 partially obstruct the flow in the gap 29 of the air sucked in by the impellers 23 and 24 or of the mixture including the fragrance emitted by the substance 16 and at least part of the sucked air, they are appropriately retracted (toward the longitudinal axis of the wall 3) with respect to the mouth 20 so as to allow the air or the aforesaid mixture to exit the diffuser 1 substantially at the whole mouth 20 (“bypassing” the segments of the guides 38 passing through the gap 29).

Similarly to what has been mentioned for the motors of the impellers 23 and 24, the control unit is connected to the actuator 36 and is suitable for controlling both an operation and a stop thereof. A user of the diffuser 1 can control the actuator 36, by means of the control unit, by means of the button 34 (or the buttons 34 if more than one). The actuator 23 is further preferably powered by means of the previously mentioned electric accumulator.

As previously mentioned, the top wall 5 of the diffuser 1 is convex with the concavity facing the base 2. The diffuser 1 preferably comprises one or more de-vices, preferably housed in the vault 4, suitable for projecting on the wall 5 visual information capturable by a user of the diffuser 1 when observing the wall 5. Therefore, the latter is preferably at least partially permeable to light.

Similarly to what has been mentioned for the motors of the impellers 23 and 24 and for the actuator 36, the control unit is connected to said projection devices and is suitable for controlling both an operation and a stop thereof. The aforesaid projection devices are further preferably powered by means of the previously mentioned electric accumulator. The control unit is preferably suitable for projecting information on the wall 5 regarding the operating state of the impellers 23 and 24, the residual charge amount of the electric accumulator and the flow rate at which the impellers 23 and 24 suck air from the environment surrounding the diffuser 1 into the cavity 7.

The container 8 preferably comprises at least a first radio-frequency electro-magnetic wave transceiver suitable for both receiving at least a first predefined signal and emitting, upon receiving the aforesaid first signal, at least a second signal, also predefined. The first transceiver preferably corresponds to an RFID tag. The diffuser 1 comprises a second radio-frequency electromagnetic wave transceiver. The second transceiver preferably comprises an antenna by means of which the second transceiver is suitable for interacting with the RFID tag of the container 8 when housed in the first cavity of the diffuser 1 as previously specified. More precisely, the control unit is connected to the transceiver of the diffuser 1 and is suitable for controlling, by means of said transceiver, an emission of radio-frequency electromagnetic waves. The transceiver of the diffuser 1 is suitable for emitting first electromagnetic waves corresponding to the aforesaid first predefined signal, and is suitable for receiving second electromagnetic waves corresponding to the aforesaid second predefined signal. The container 8 is housed in the first cavity of the diffuser 1, as previously specified, so that the antenna of the transceiver is placed in the diffuser 1 in a position such that, when the transceiver of the diffuser 1 emits the aforesaid first radio-frequency electromagnetic waves by means of the aforesaid antenna:
the RFID tag of the container 8 receives the aforesaid first radio-frequency electromagnetic waves (corresponding to the aforesaid first predefined signal),
and therefore the RFID tag emits the aforesaid second radio-frequency electromagnetic waves (corresponding to the aforesaid second predefined signal) and
the transceiver of the diffuser 1 receives, by means of the aforesaid antenna, the aforesaid second radio-frequency electromagnetic waves emitted by the RFID tag of the container 8.

The aforesaid second radio-frequency electromagnetic waves emitted by the RFID tag upon an emission of the first radio-frequency electromagnetic waves by the transceiver of the diffuser 1 preferably encode information concerning the substance 16 or the fragrance which is emanated by the latter. The control unit is preferably suitable for decoding said information concerning the substance 16 or the fragrance which is emanated by the latter, from the aforesaid second radio-frequency electromagnetic waves emitted by the RFID tag of the container 8.

The transceiver antenna of the diffuser 1 is preferably at least partially, and more preferably completely, wrapped around the container 8 when the latter is housed in the first cavity of the diffuser 1 as specified above. More precisely, the aforesaid antenna is preferably shaped so as to be outlined as a circumference and is preferably housed in the wall portion 6 opposite to (i.e., surrounding) the neck 9 of the container 8 when the latter is housed in the first cavity of the diffuser 1 as specified above. Preferably, the RFID tag is applied to the container 8 at the neck 9 so that the risk is very low that the transceiver of the diffuser 1 cannot interact with the RFID tag of the container 8 when the latter is housed in the first cavity of the diffuser 1 as previously specified (i.e., with the neck 9 screwed into the wall 6 at the opening 15).

Similarly to what has been mentioned for the motors of the impellers 23 and 24, the actuator 36 and the projection devices, the transceiver of the diffuser 1 is preferably powered by means of the previously mentioned electric accumulator. The control unit is preferably suitable for operating the aforementioned projection devices so as to project onto the wall 5, and therefore show to a user of the diffuser 1 observing the wall 5, the aforementioned information concerning the substance 16 or the fragrance which is emanated by the latter, and decoded by the aforesaid second radio-frequency electromagnetic waves emitted by the RFID tag of the container 8.

When the diffuser 1 is not in use, the vault 4 is preferably in the closed position and the impellers 23 and 24 are stopped. When a user, by means of the button 34, turns on the diffuser 1, the control unit moves the vault 4 from the closing position to the opening position and operates the motors of the impellers 23 and 24. Therefore, the diffuser 1 begins to emit from the mouth 20 a mixture of air (corresponding to at least part of the air sucked by the impellers 23 and 24 into the portion 32 of the cavity 7) and fragrance emanated by the substance 16 included in the cavity 13 of the container 8. Preferably, the control unit is suitable for switching off the diffuser 1 (stopping the rotation of the impellers 23 and 24 and then translating the vault 4 from the opening position to the closing position) after a given period of operation (for example ten minutes).

According to a variant of the diffuser 1, the latter comprises the container 8 at least partially housed in the first cavity of the diffuser 1 and reversibly connected to the wall 6 thereof at its neck 9 as previously described. Incidentally, asserting that the container 8 is reversibly connected to the diffuser 1 means that the container 8 is disconnectable from the diffuser 1 and reconnectable thereto an indefinite number of times.

Based on the description provided for a preferred embodiment, it is apparent that some changes may be introduced by those skilled in the art without departing from the scope of the invention as defined by the following claims.

1-14. (canceled)

15. A room fragrance diffuser,
said diffuser comprising:
a base at which said diffuser is placeable on a surface;
one or more peripheral walls;
a first cavity accessible, from the environment surrounding said diffuser, through a first opening placed at said base or said peripheral wall or at least one of said peripheral walls if more than one;
a second cavity accessible, from the environment surrounding said diffuser, through at least one inlet mouth and at least one outlet mouth,
said inlet mouth being placed at said peripheral wall or at least one of said peripheral walls if more than one,
said outlet mouth being placed at said peripheral wall or at least one of said peripheral walls if more than one,
said first cavity and said second cavity being separated from each other by at least one inner wall of said diffuser,
said first cavity and said second cavity being in communication with each other through a second opening placed at said inner wall;
gas-operated means suitable for
sucking air from the environment surrounding said diffuser into said second cavity through said inlet mouth and for
expelling into the environment surrounding said diffuser, through said outlet mouth, at least part of the air sucked into said second cavity,
so that at least part of the air sucked into said second cavity flows into the latter from said inlet mouth toward said outlet mouth,
said second cavity being therefore travelable by air;
control means connected to said gas-operated means and suitable for controlling both an operation and a stop of said gas-operated means;
a container housed, at least partially, at said first cavity,
said container being removable from said first cavity and then at least partially housable in said first cavity through said first opening,
said container comprising a cavity delimited by at least one wall,
said cavity of said container being accessible through an opening placed at said wall of said container or at least one of said walls of said container if more than one,
said container being suitable for housing, in its cavity, an amount of a substance,
said substance housable in said cavity of said container being suitable for emanating a fragrance when:
said substance is housed in said cavity of said container so as to comprise at least one free surface thereof and
air in contact with said free surface is sucked away from said free surface,
said fragrance, when emanated, being emanated by said substance at said free surface,
said container being at least partially housed in said first cavity so that said opening of said container is in communication with said second opening of said diffuser,
so that said cavity of said container is in communication with said second cavity of said diffuser through said opening of said container and said second opening of said diffuser,
said diffuser being wherein said outlet mouth is substantially annular,
said outlet mouth being delimited by a first edge and a second edge opposite to said first edge,
each of said first and second edges extending in length so as to form a closed path,
said second cavity, at least at a portion thereof adjacent to said outlet mouth, being interposed between a first wall and a second wall,
said first wall extending from said first edge to a third edge extending in length so as to form a closed path,
said second wall extending from said second edge to a fourth edge extending in length so as to form a closed path,
said first wall being at least partially surrounded by said second wall so that said portion of said second cavity corresponds to a gap interposed between said first wall and said second wall,
said gap having a converging trend away from said outlet mouth,
said diffuser comprising at least two of said inlet mouths,
said second cavity comprises a section at which said second cavity is in communication:
with the environment surrounding said diffuser through said two inlet mouths, said two inlet mouths lying on opposite sides with respect to said section of said second cavity,
with said cavity of said container through said opening of said container and said second opening of said diffuser and
with said gap through a third opening delimited by said third and fourth edges,
said third opening extending in length and being shaped as a circular crown,
said second opening and said third opening lying on opposite sides with respect to said section of said second cavity,
when said diffuser is placed on a surface at said base:
said second opening lying at a lower height than that at which said third opening lies and
each of said two inlet mouths lying at a lower height than that at which said third opening lies,
said gas-operated means comprising at least two vane impellers,
said two vane impellers lying on opposite sides with respect to said section of said second cavity,
each of said two vane impellers being opposite to one of said two inlet mouths,
each of said two vane impellers being housed in said second cavity and being oriented so that the air sucked from the environment surrounding said diffuser, when sucked by said vane impeller into said second cavity, is at least partially sucked into said section of said second cavity and blown toward said third opening,
when said two vane impellers suck air into said second cavity from the environment surrounding said diffuser, the air sucked into said second cavity, while it travels through the latter towards said outlet mouth, flowing from said section of said second cavity into said gap of said second cavity, the cross-section of said second cavity,
at said section of said second cavity and at said gap of said second cavity, decreasing,
with reference to the air flow direction when said two vane impellers suck air into said second cavity from the environment surrounding said diffuser,
in a segment of said second cavity including said third opening, that is in a segment of said second cavity including a passage from said section of said second cavity to said gap of said second cavity,
when said two vane impellers suck air into said second cavity from the environment surrounding said diffuser, the air accelerating by traveling through said segment of said second cavity so that, at said segment of said second cavity, a decrease in air pressure occurs by Venturi effect,
said segment of said second cavity being sufficiently close to said second opening so that, when said two vane impellers suck air into said second cavity from the environment surrounding said diffuser, said decrease in air pressure causes a suction of air from said cavity of said container into said second cavity through said second opening,
so that, when said two vane impellers suck air into said second cavity from the environment surrounding said diffuser and said substance is housed in said cavity of said container, said decrease in pressure of the air sucked into said second cavity at said segment of said second cavity causes:
a movement away from said free surface of air in contact therewith
and therefore an emanation of said fragrance from said free surface
and therefore a suction of said fragrance from said cavity of said container into said second cavity of said diffuser through said opening of said container and said second opening of said diffuser
and therefore a mixing of said fragrance with at least part of the air sucked into said second cavity by said two vane impellers
and therefore an expulsion into the environment surrounding said diffuser, through said outlet mouth, of said mixture including said fragrance and at least part of the air sucked into said second cavity,
the fact that said outlet mouth is substantially annular and the converging trend of said gap of said second cavity away from said outlet mouth meaning that, when said two vane impellers suck air into said second cavity from the environment surrounding said diffuser, air sucked into said second cavity, or said mixture including said fragrance and at least part of the air sucked into said second cavity, is expelled into the environment surrounding said diffuser, through said outlet mouth, directed so as to give rise to a flow divergent away from said diffuser all around the latter and substantially planar or at least partially frustoconical at least at a portion of said environment surrounding said diffuser adjacent to said outlet mouth.

16. The diffuser according to claim 15, wherein said outlet mouth is arranged horizontally when said diffuser is placed on a surface at said base, so that, said flow with which air sucked into said second cavity, or said mixture including said fragrance and at least part of the air sucked into said second cavity, is expelled into the environment surrounding said diffuser all around the latter when said gas-operated means suck air from the environment surrounding said diffuser into said second cavity, is substantially, at least at said portion of said environment surrounding said diffuser adjacent to said outlet mouth:
planar and substantially arranged horizontally or
at least partially frustoconical with substantially vertical axis when said diffuser is placed on a surface at said base.

17. The diffuser according to claim 15, wherein it comprises reversible closing means of said outlet mouth.

18. The diffuser according to claim 17, wherein said reversible closing means comprise:
a shutter including said first edge or said second edge;
movement means suitable for moving said shutter between at least:
a first opening position, at which said first and second edges are not in contact with each other so that, when said two vane impellers suck air into said second cavity from the environment surrounding said diffuser, air sucked into said second cavity, or said mixture including said fragrance and at least part of the air sucked into said second cavity, can exit from said diffuser through said outlet mouth and
a second closing position, at which said first and second edges are in contact with each other so as to occlude said outlet mouth so that, when said two vane impellers suck air into said second cavity from the environment surrounding said diffuser, air sucked into said second cavity, or said mixture including said fragrance and at least part of the air sucked into said second cavity, cannot exit from said diffuser through said outlet mouth,
said control means being connected to said movement means and being suitable for controlling both an operation and a stop of said movement means,
said first wall extending from said first edge at least when said shutter is in said first position,
said second wall extending from said second edge at least when said shutter is in said first position.

19. The diffuser according to claim 18, wherein said control means are suitable for preventing said two vane impellers from sucking air into said second cavity from the environment surrounding said diffuser when said shutter is in said second position.

20. The diffuser according to claim 18, wherein said first wall at least partially delimits a third cavity surrounded by said gap with the interposition of said first wall,
said movement means being at least partially housed in said third cavity.

21. The diffuser according to claim 15, wherein it comprises:
a top wall opposite to said base;
display means suitable for displaying visual information at said top wall so that said information is capturable by a person when observing said top wall,
said control means being connected to said display means and being suitable for controlling them.

22. The diffuser according to claim 21, wherein said top wall is at least partially convex with the concavity facing said base.

23. The diffuser according to claim 21, wherein said display means are suitable for displaying, at said top wall, information concerning the flow rate at which said two vane impellers suck air from the environment surrounding said diffuser into said second cavity through said inlet mouths.

24. The diffuser according to claim 15, wherein said container comprises at least one radio-frequency electromagnetic wave transceiver suitable for both receiving at least a first predefined signal and emitting, upon receiving said first signal, at least a second signal, also predefined,
said diffuser comprising radio-frequency electromagnetic wave transceiver means,
said control means being connected to said transceiver means and being suitable for controlling, by means of said transceiver means, an emission of radio-frequency electromagnetic waves,
said transceiver means being suitable for emitting first radio-frequency electromagnetic waves corresponding to said first signal,
said transceiver means being suitable for receiving second radio-frequency electromagnetic waves corresponding to said second signal,
said container being housed in said first cavity so that, and said transceiver means being placed in said diffuser so that, when said transceiver means emit said first radio-frequency electromagnetic waves:
said transceiver receives said first radio-frequency electromagnetic waves
and therefore said transceiver emits said second radio-frequency electromagnetic waves and
said transceiver means receiving said second radio-frequency electromagnetic waves emitted by said transceiver,
said second radio-frequency electromagnetic waves emitted by said transceiver upon an emission of said first radio-frequency electromagnetic waves by said transceiver means encoding information concerning said fragrance being emanable by said substance housable in said cavity of said container,
said control means being suitable for decoding, from said second radio-frequency electromagnetic waves emitted by said transceiver, said information concerning said fragrance being emanable by said substance housable in said cavity of said container.

25. The diffuser according to claim 24, wherein said transceiver means comprise an antenna for transceiving radio-frequency electromagnetic waves arranged so that said antenna is at least partially wrapped around said container when the latter is housed in said first cavity.

26. The diffuser according to claim 24, wherein said display means are suitable for displaying, at said top wall, said information concerning said fragrance being emanable by said substance housable in said cavity of said container decoded by said second radio-frequency electromagnetic waves emitted by said transceiver upon an emission of said first radio-frequency electromagnetic waves by said transceiver means.

27. The diffuser according to claim 15, wherein it comprises electricity storage means,
said control means, said two vane impellers, said movement means, if present, said display means, if present, and said transceiver means, if present, being connected to said storage means for powering them,
said display means, if present, being suitable for displaying, at said top wall, information concerning the amount of electricity present in said storage means.

28. The diffuser according to claim 15, wherein said base is circular and said peripheral wall corresponds to, or said peripheral walls, if more than one, comprise, a cylindrical or frustoconical side wall connected to said base.
